(19) United States
(12) Patent Application Publication
(10) Pub. No.: US 2024/0138916 A1
(43) Pub. Date: May 2, 2024

(54) LASER TRAJECTORY MARKER

(71) Applicant: Medos International Sarl, LeLocie (CH)

(72) Inventors: Jorn Richter, Oberdorf (CH); Daniela Wehrli, Raynham, MA (US)

(21) Appl. No.: 18/061,760

(22) Filed: Dec. 5, 2022

Related U.S. Application Data
(60) Provisional application No. 63/381,079, filed on Oct. 26, 2022.

(51) Int. Cl. A61B 34/10; A61B 17/3211; A61B 34/30

(52) U.S. Cl. CPC A61B 34/10; A61B 17/3211; A61B 34/30; A61B 2090/3937

(57) ABSTRACT

Systems, methods, and devices are disclosed for robotic surgical systems including a non-contact marking device, comprising a body sized and shaped to be at least partially inserted into a guide slot of a scalpel guide; a shoulder extending outwardly from the body configured to limit a distance the body may be at least partially inserted into the guide slot of the scalpel guide; and a marking device configured to emit a light, the marking device configured to project light from a bottom face of the body such that the light projected by the marking device is coaxially aligned with a longitudinal axis of a tool guide of a surgical system when the non-contact marking device is at least partially inserted in the guide slot of the scalpel guide while the scalpel guide is inserted in the tool guide of the surgical system.

### INCORPORATION BY REFERENCE

The present patent application claims priority to the provisional application U.S. Ser. No. 63/381,079, filed on Oct. 26, 2022; the entire contents of which are hereby expressly incorporated herein by reference.

### BACKGROUND

A computer-assisted surgical system may include a robotic arm, controller, and navigational system. Robotic or robot-assisted surgeries have many associated advantages, particularly in terms of precise placement of surgical tools and/or implants. For example, during robot-assisted spine surgery, a trajectory is planned for a tool or series of tools attached to the robotic arm via a tool guide based on a surgical plan. During surgery, once the robotic arm has guided the tool guide to the planned trajectory, a first interaction with a patient is for a surgeon to create a skin incision at the intersection of the planned trajectory and the skin. Generally, this is done with a simple stab incision through a scalpel guide placed in the tool guide. However, the incision is typically made longer than a diameter of the tool guide, which is why the surgeon must manually enlarge the initial stab incision that was done through the scalpel guide.

### SUMMARY

To overcome the need for this two-step incision process, the presently disclosed systems, devices, and methods improve computer-assisted surgical systems, for instance, by providing a non-contact mark projected along the planned trajectory to the patient's skin to allow the surgeon to make a single incision.

Systems, methods, and devices are described for robotic surgical systems. Some embodiments of the invention provide a surgical robot (and optionally a navigation system) that utilizes a positioning system that allows movement of a tool guide to a planned trajectory where a longitudinal axis of the tool guide is coaxially aligned with the planned trajectory and a non-contact marking device placed in the tool guide marks an incision point on a skin of a patient at an intersection of the planned trajectory and the skin. In some embodiments, a robotic surgical system may be provided with a surgical robot having a base, a robotic arm coupled to and configured for articulation relative to the base, a tool guide coupled to a distal end of the robotic arm, a scalpel guide having a guide slot adapted for receiving a scalpel secured in the tool guide, and a non-contact marking device configured to be at least partially inserted into the guide slot and project mark using a light visible to human such as a laser.

In some embodiments a non-contact marking device may be provided, comprising: a body having a bottom face, a first face, a second face separated a first predetermined distance from the first face, a first side, and a second side separated a second predetermined distance from the first side, the body sized and shaped to be at least partially inserted into a guide slot of a scalpel guide; a shoulder extending outwardly from the body and formed a predetermined distance from the bottom face of the body and configured to limit a distance the body may be at least partially inserted into the guide slot of the scalpel guide; and a marking device configured to emit a light, the marking device configured to project light from the bottom face of the body such that the light projected by the marking device is coaxially aligned with a longitudinal axis of a tool guide of a surgical system when the non-contact marking device is at least partially inserted in the guide slot of the scalpel guide while the scalpel guide is inserted in the tool guide of the surgical system.

The non-contact making device may be provided with a button electrically connected to the marking device and configured to switch the marking device from an off state wherein the marking device does not emit light to an on state wherein the marking device emits the light.

Also described is a non-contact marking device that may be provided, with a body having a lower face and an upper face, the body sized and shaped to be at least partially inserted into an aperture of a tool guide of a surgical system, the body having a guide slot formed in and extending through the body from the upper face to the lower face, the guide slot sized and shaped to receive a body of a scalpel, the body having; a shoulder extending outwardly from the body; a top having an upper surface and a lower surface spaced a predetermined distance apart and connected to at least one of the body and the shoulder; a marking device supported by the top configured to emit a light through the guide slot, the marking device disposed in the lower surface of the top; and a button electrically connected to the marking device and configured to switch the marking device from an off state wherein the marking device does not emit light to an on state wherein the marking device emits the light.

The non-contact marking device may further be provided wherein the top is hingedly connected to the shoulder such that the top may be moved between an open position and a closed position.

The non-contact marking device may further be provided wherein the button is supported by the lower surface of the top and when the top is in the closed position the lower surface of the top and the button are in contact with the top face of the shoulder and the marking device is in the on state.

Also described is a robotic surgical system comprising: a robotic arm; a tool guide supported by the robotic arm, the tool guide comprising a tool support having a first end, a second end, an aperture extending through the tool support from the first end to the second end, and a longitudinal axis extending through a center of the aperture from the first end to the second end; and a controller in communication with the robotic arm, the controller having a non-transitory computer readable memory and a processor, the non-transitory computer readable memory storing at least one planned trajectory associated with a surgical procedure and processor executable instructions that, when executed, cause the processor to pass a first signal to the robotic arm causing the robotic arm to position the tool support a distance from a patient with the longitudinal axis of the tool support substantially coaxially aligned with the at least one planned trajectory; and a non-contact marking device comprising: a body having a first body portion having a first diameter extending from a bottom face to a first shoulder and a second body portion having a second diameter that is larger than the first diameter extending from the first shoulder to a second shoulder, the body positioned within the aperture of the tool support, the body having a central axis; and a marking device configured to emit a light in a wavelength visible to a human eye, the marking device configured to emit light from the bottom face of the body such that the marking device is coaxially aligned with the central axis extending through a center of the body.

The robotic surgical system may be provided further comprising a button electrically connected to the marking device and configured to switch the marking device from an off state wherein the marking device does not emit light to an on state wherein the marking device emits the light.

The robotic surgical system may be provided wherein the body of the non-contact marking device further comprises a third body portion having a third diameter that is larger than the second diameter, the third body portion extending from the second shoulder to a third shoulder.

### BRIEF DESCRIPTION OF THE DRAWINGS

FIG. 1 shows a schematic of a computer-assisted surgical system including a robot base, a robotic arm, a tool guide attached to the robotic arm, a scalpel guide having a guide slot adapted for receiving a scalpel secured in the tool guide, and a non-contact marking device configured to be inserted into the guide slot and project a mark in accordance with one embodiment of the present disclosure;

FIG. 2 is an exploded, perspective view of the tool guide, the scalpel guide having the guide slot adapted for receiving a scalpel, and the non-contact marking device configured to be inserted into the guide slot and project a mark of FIG. 1;

FIGS. 3A-3C are perspective views of another scalpel guide having a non-contact marking device configured to project a mark hingedly connected to the scalpel guide constructed in accordance with one embodiment of the present disclosure;

FIGS. 4A and 4B are perspective views of another non-contact marking device configured to project an indicator having a stepped body with a first portion of the stepped body having a first diameter, a second portion of the stepped body having a second diameter greater than the first diameter, and a third portion of the stepped body having a third diameter greater than the second diameter constructed in accordance with one embodiment of the present disclosure.

FIG. 5 shows a workflow for making an incision for a surgical procedure which employs a computer-assisted surgical system using a non-contact marking device in accordance with one embodiment of the present disclosure.

### DETAILED DESCRIPTION

Before explaining at least one embodiment of the disclosure in detail, it is to be understood that the disclosure is not limited in its application to the details of construction, experiments, exemplary data, and/or the arrangement of the components set forth in the following description or illustrated in the drawings unless otherwise noted.

The systems and methods as described in the present disclosure are capable of other embodiments or of being practiced or carried out in various ways. Also, it is to be understood that the phraseology and terminology employed herein is for purposes of description, and should not be regarded as limiting.

The following detailed description refers to the accompanying drawings. The same reference numbers in different drawings may identify the same or similar elements.

As used in the description herein, the terms “comprises,” “comprising,” “includes,” “including,” “has,” “having,” or any other variations thereof, are intended to cover a non-exclusive inclusion. For example, unless otherwise noted, a process, method, article, or apparatus that comprises a list of elements is not necessarily limited to only those elements, but may also include other elements not expressly listed or inherent to such process, method, article, or apparatus.

Further, unless expressly stated to the contrary, “or” refers to an inclusive and not to an exclusive “or”. For example, a condition A or B is satisfied by one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more, and the singular also includes the plural unless it is obvious that it is meant otherwise. Further, use of the term “plurality” is meant to convey “more than one” unless expressly stated to the contrary.

As used herein, any reference to “one embodiment,” “an embodiment,” “some embodiments,” “one example,” “for example,” or “an example” means that a particular element, feature, structure or characteristic described in connection with the embodiment is included in at least one embodiment. The appearance of the phrase “in some embodiments” or “one example” in various places in the specification is not necessarily all referring to the same embodiment, for example.

Circuitry, as used herein, may be analog and/or digital components, or one or more suitably programmed processors (e.g., microprocessors) and associated hardware and software, or hardwired logic. Also, “components” may perform one or more functions. The term “component” may include hardware, such as a processor (e.g., microprocessor), a combination of hardware and software, and/or the like. Software may include one or more computer executable instructions that when executed by one or more components cause the component to perform a specified function. It should be understood that the algorithms described herein may be stored on one or more non-transitory memory. Exemplary non-transitory memory may include random access memory, read only memory, flash memory, and/or the like. Such non-transitory memory may be electrically based, optically based, and/or the like.

As used herein, the term “substantially” means that the subsequently described event or circumstance completely occurs or that the subsequently described event or circumstance occurs to a great extent or degree. As used herein the qualifier “substantially” is intended to include not only the exact value, amount, degree, orientation, or other qualified characteristic or value, but are intended to include some slight variations due to measuring error, control loop error, manufacturing tolerances, stress exerted on various parts or components, observer error, wear and tear, and combinations thereof. For example, when describing the longitudinal axis of the tool support substantially coaxially aligned with at least one planned trajectory, the term “substantially” refers to alignment within tracking tolerances.

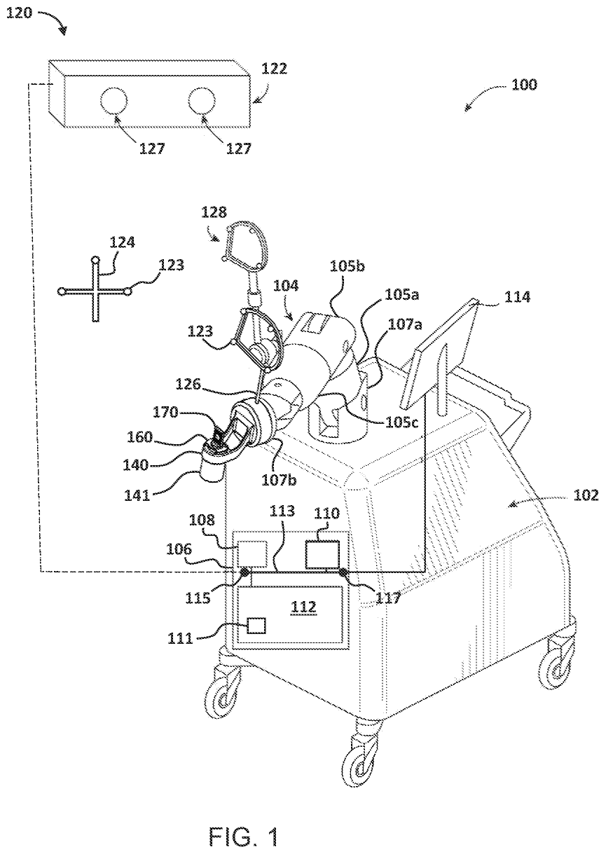

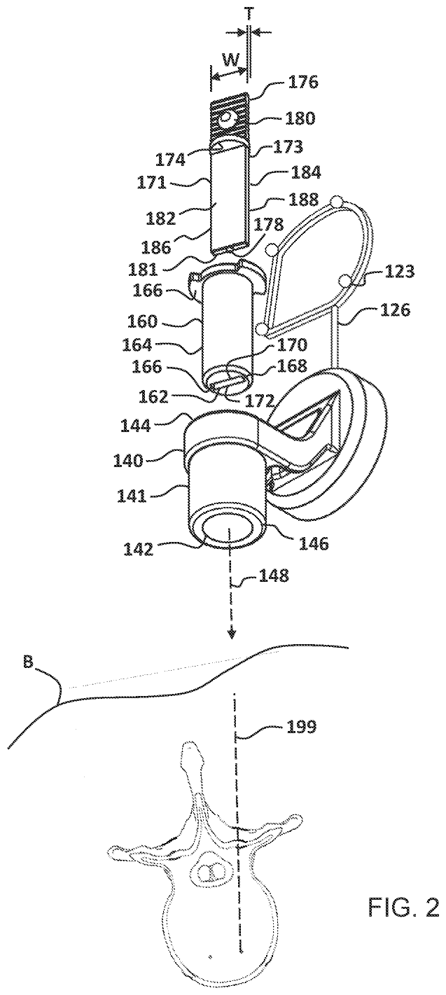

Referring now to the drawings, and in particular to FIGS. 1 and 2, shown therein is an overview of an exemplary computer-assisted surgical system 100. The computer-assisted surgical system 100 may be provided with a surgical robot 101 having a robot base 102 supporting a robotic arm 104 and a navigation system 120. A tool guide 140 may be attached to the robotic arm 104 and be configured to receive a scalpel guide 160 and a non-contact marking device 170 in accordance with the present disclosure.

The robot base 102 is depicted as a mobile base, but stationary bases are also contemplated. The robotic arm 104 includes a plurality of arm segments 105a, 105b, 105c connected by rotatable or otherwise articulating joints and may be moved by actuation of the joints. One of the arms 105 forms a distal end 107b of the robotic arm 104. In the example shown in FIG. 1, the robotic arm 105c of the robotic arm 104 forms the distal end 107b. The robotic arm 104 also includes a proximal end 107a attached to and supported by the robot base 102, and the distal end 107b. The robotic arm 104 may be adapted to move in all six degrees of freedom during a surgical procedure. The robotic arm 104 may be configured for incremental changes (e.g., in each of the six degrees of freedom) to ensure the necessary precision during surgery. The robotic arm 104 may actively move about the joints to position the robotic arm 104 in a desired position relative to a patient (not depicted), or the robotic arm 104 may be set and locked into a position. For example, the present disclosure is contemplated to include use of tools by surgical robots, by users with some degree of robotic assistance, and without involvement of surgical robots or robotic assistance (e.g., once positioned and locked).

A control unit or controller 106 enables various features of the system 100, and performance of various methods disclosed herein in accordance with some embodiments of the present disclosure. In some embodiments, the controller 106 can control operation of the robotic arm 104 and associated navigational system(s) 120. In some embodiments, the control may comprise calibration of relative systems of coordinates, generation of planned trajectories, monitoring of position of various units of the surgical robot 101, and/or units functionally coupled thereto, implementation of safety protocols or limits, and the like. The controller 106 may be a system or systems able to embody and/or execute logic of processes described herein. The controller 106 may be configured to execute logic embodied in the form of software instructions and/or firmware. In some embodiments, the logic described herein may be executed in a stand-alone environment such as on the controller 106 and/or logic may be implemented in a networked environment such as a distributed system using multiple computers and/or processors.

The various embodiments of the present disclosure can be operational with other computing systems, environments, and/or configurations that can be suitable for use with the systems and methods of the invention comprise personal computers, server computers, laptop devices or handheld devices, and multiprocessor systems configured to execute logic embodied in the form of software instructions and/or firmware described herein. Additional examples comprise mobile devices, programmable consumer electronics, network PCs, minicomputers, mainframe computers, distributed computing environments that comprise any of the above systems or devices, and the like.

The controller 106 may include one or more processors 108 (hereinafter “processor 108”), one or more communication devices 110 (hereinafter “communication device 110”), one or more non-transitory memory 112 (hereinafter “memory 112”) storing processor executable code and/or software application(s), such as application 111, and a system bus 113 that couples various components including the processor 108 to the memory 112, for example.

In general, the processor 108 refers to any computing processing unit or processing device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, or alternatively, the processor 108 may be an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors or processing units referred to herein can exploit nano-scale architectures such as, molecular and quantum-dot based transistors, switches and gates, in order to optimize space usage or enhance performance of the computing devices that can implement the various aspects of the subject invention. In some embodiments, processor 108 also can be implemented as a combination of computing processing units.

An external device 114 may communicate with the controller 106. The external device 114 may be a touch-screen display, a computing device, remote server, etc., configured to allow a surgeon or other user to input data directly into the controller 106. Such data may include patient information and/or surgical procedure information. The external device 114 may display information from the controller 106, such as alerts. Communication between the external device 114 and the controller 106 may be wireless or wired. The illustrated external device 114 is shown attached to the robot base 102, however, in some embodiments, the external device 114 may be portable and placed in various locations within an operating room.

The system 100 may also comprise a navigational system 120 that includes a tracking unit 122. The system 100 is able to monitor, track, and/or determine changes in the relative position and/or orientation of one or more parts of the robotic arm 104, the tool guide 140, and/or a tool inserted in the tool guide 140, as well as various parts of the patient's body B, within a common coordinate system by utilizing various types of fiducials 123 (e.g., multiple degree-of-freedom optical, inertial, and/or ultrasonic sensing devices), navigation systems (e.g., machine vision systems, charge coupled device cameras, tracker sensors, surface scanners, and/or range finders), anatomical computer models (e.g., magnetic resonance imaging scans of the lower lumbar region of the spine), data from previous surgical procedures and/or previously-performed surgical techniques (e.g., data recorded by the system 100 while performing earlier steps of a surgical procedure), and the like. Tracking may be performed in a number of ways, e.g., using stereoscopic optical detectors 127, ultrasonic detectors, sensors configured to receive position information from inertial measurement units, etc. Tracking in real time, in some embodiments, means high frequencies greater than twenty Hertz, in some embodiments in the range of one hundred to five hundred Hertz, with low latency, in some embodiments less than five milliseconds. Regardless of how it is gathered, position and orientation data may be transferred between components (e.g., to the controller 106) via any suitable connection, e.g., with wires or wirelessly using a low latency transfer protocol. The controller 106 may carry out real-time control algorithms at a reasonably high frequency with low additional latency to coordinate movement of the robotic arm 104 of the system 100. The tracking unit 122 may also include cameras, or use the stereoscopic optical detectors 127, to detect, for example, characteristics of the tool guide 140 attached to the robotic arm 104.

Fiducials 123 of the navigational system 120 may be attached to the navigation arrays (e.g., a first navigation array 124, a second navigational array 126, and an optional navigation array 128 (and/or other navigation arrays)). Fiducials 123 may be arranged in predetermined positions and orientations with respect to one another. The fiducials 123 may be aligned to lie in planes of known orientation (e.g., perpendicular planes, etc.) to enable setting of a Cartesian reference frame. The fiducials 123 may be positioned within a field of view of a navigation system 120 and may be identified in images captured by the navigation system 120. The fiducials 123 may be single-use reflective navigation markers. Exemplary fiducials 123 include infrared reflectors, light emitting diodes (LEDs), spherical reflective markers, blinking LEDs, augmented reality markers, and so forth. The first navigation array 124, second navigation array 126, and optional navigation array 128 may be or may include an inertial measurement unit (IMU), an accelerometer, a gyroscope, a magnetometer, other sensors, or combinations thereof. The sensors may transmit position and/or orientation information to the navigation system 120. In other embodiments, the sensors may be configured to transmit position and/or orientation information to an external controller which may be, for example, the controller 106.

The second navigation array 126 may be mounted on the robotic arm 104 or on the tool guide 140 and may be used to determine a position of the robotic arm 104 or a distal portion thereof (indicative of a position of the tool guide 140). The structure and operation of the second navigation array 126 may vary depending on the type of navigation system 120 used. In some embodiments, the second navigation array 126 may include one or more sphere-shaped or other fiducials 123 for use with an optical navigation system, for example, the second navigation array 126 illustrated in FIG. 2 with the spherical fiducial 123. The navigation system 120 facilitates registering and tracking of the position and/or orientation of the second navigation array 126 and, by extension, the tool guide 140 and a relative distance of the tool guide 140 to other objects in the operating room, e.g., a patient, a surgeon, etc. Position and/or orientation data may be gathered, determined, or otherwise handled by the navigation system 120 using registration/navigation techniques to determine coordinates of each navigation array and/or fiducial 123 within a coordinate system. These coordinates may be communicated to the controller 106 which uses the coordinates of each navigation array and/or fiducial 123 to calculate a position and orientation of the tool guide 140 in the coordinate system and a position of the tool guide 140 relative to the patient to facilitate articulation of the robotic arm 104.

The application 111 may configure the controller 106, or the processor 108 thereof, to perform the automated control of position of the robotic arm 104 in accordance with aspects of the invention. Such control can be enabled, at least in part, by the navigation system 120. In some embodiments, when the controller 106 is functionally coupled to the robotic arm 104, the application 111 can configure the controller 106 to perform the functionality described in the present disclosure. In some embodiments, the application 111 may be retained or stored in memory 112 as a group of computer-accessible instructions (for instance, computer-readable instructions, computer-executable instructions, or computer-readable computer-executable instructions). In some embodiments, the group of computer-accessible instructions can encode the methods of the presently disclosed inventive concepts. In some embodiments, the application 111 may encode various formalisms (e.g., image segmentation) for computer vision tracking using the navigation system 120. In some embodiments, the application 111 may be a compiled instance of such computer-accessible instructions stored in the memory 112, a linked instance of such computer-accessible instructions, a compiled and linked instance of such computer-executable instructions, or an otherwise executable instance of the group of computer-accessible instructions.

The memory 112 may be any available media that is accessible by the controller 106 and comprises, for example and not meant to be limiting, both volatile and/or non-volatile media, removable and/or non-removable media. In some embodiments, the memory 112 comprises computer readable media in the form of volatile memory, such as random access memory (RAM), and/or non-volatile memory, such as read only memory (ROM). In some embodiments, the memory 112 may store data (such as a group of tokens employed for code buffers) and/or program modules such as the application 111 that are immediately accessible to, and/or are presently operated-on by the controller 106. In some embodiments, the memory may store an operating system (not shown) such as Windows operating system, Unix, Linux, Symbian, Android, Apple iOS operating system, Chromium, and substantially any operating system for wireless computing devices or tethered computing devices. Apple® is a trademark of Apple Computer, Inc., registered in the United States and other countries. iOS® is a registered trademark of Cisco and used under license by Apple Inc. Microsoft® and Windows® are either registered trademarks or trademarks of Microsoft Corporation in the United States and/or other countries. Android® and Chrome® operating system are registered trademarks of Google Inc. Symbian® is a registered trademark of Symbian Ltd. Linux® is a registered trademark of Linus Torvalds. UNIX® is a registered trademark of The Open Group.

In some embodiments, the memory 112 may be a mass storage device which can provide non-volatile storage of computer code (e.g., computer-executable instructions such as the application 111), computer-readable instructions, data structures, program modules, and other data for the controller 106. For instance, in some embodiments, the memory 112 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

In some embodiments, optionally, any number of program modules can be stored on the memory 112, including by way of example, the operating system, and a tracking software (not shown). In some embodiments, data and code (for example, computer-executable instructions, patient-specific trajectories, and patient anatomical data) may be retained and stored on the memory 112. In some embodiments, data and/or code, may be stored in any of one or more databases known in the art. Examples of such databases comprise, DB2®, Microsoft® Access, Microsoft® SQL Server, Oracle®, mySQL, PostgreSQL, and the like. Further examples include membase databases and flat file databases. The databases can be centralized or distributed across multiple systems.

DB2® is a registered trademark of IBM in the United States.

Microsoft®, Microsoft® Access®, and Microsoft® SQL Server™ are either registered trademarks or trademarks of Microsoft Corporation in the United States and/or other countries.

Oracle® is a registered trademark of Oracle Corporation and/or its affiliates.

MySQL® is a registered trademark of MySQL AB in the United States, the European Union and other countries.

PostgreSQL® and the PostgreSQL® logo are trademarks or registered trademarks of The PostgreSQL Global Development Group, in the U.S. and other countries.

In some embodiments, the user (for example, a surgeon or other user, or equipment) can enter commands and information into the controller 106 via the external device 114 using an input device (not shown). Examples of such input devices include, but are not limited to, a keyboard, a pointing device (for example, a mouse), a microphone, a joystick, a scanner (for example, a barcode scanner), a reader device such as a radiofrequency identification (RFID) readers or magnetic stripe readers, gesture-based input devices such as tactile input devices (for example, touch screens, gloves and other body coverings or wearable devices), speech recognition devices, or natural interfaces, and the like.

In some embodiments, the external device 114 may be functionally coupled to the system bus 113 via an interface 116. In some embodiments, the controller 106 may be configured to have more than one external device 114. For example, in some embodiments, the external device 114 may be a monitor, a liquid crystal display, or a projector. Further, in addition to the external device 114, some embodiments may include other output peripheral devices that can comprise components such as speakers (not shown) and a printer (not shown) capable of being connected to the controller 106 via interface 116. In some embodiments, a pointing device, may be either tethered to, or wirelessly coupled to the controller 106 to receive input from the user. In some embodiments, any step and/or result of the methods can be output in any form to an output device such as the external device 114. In some embodiments, the output can be any form of visual representation, including, but not limited to, textual, graphical, animation, audio, tactile, and the like.

In certain embodiments, one or more cameras may be contained or functionally coupled to the navigation system 120, which is functionally coupled to the system bus 113 via an input/output interface 115. Such functional coupling can permit the one or more camera(s) to be coupled to other functional elements of the controller 106. In one embodiment, the input/output interface 115, at least a portion of the system bus 113, and the memory 112 can embody a frame grabber unit that can permit receiving imaging data acquired by at least one of the one or more cameras. In some embodiments, the frame grabber can be an analog frame grabber, a digital frame grabber, or a combination thereof. In some embodiments, where the frame grabber is an analog frame grabber, the processor 108 can provide analog-to-digital conversion functionality and decoder functionality to enable the frame grabber to operate with medical imaging data. Further, in some embodiments, the input/output interface 115 can include circuitry to collect the analog signal received from at least one camera of the one or more cameras. In some embodiments, in response to execution by processor 108, the application 111 may operate the frame grabber to receive imaging data in accordance with various aspects described herein.

The tool guide 140 may be coupled to the robotic arm 104 using conventional means known in the art. As can appreciated, there should be no play between the tool guide 140 and robotic arm 104.

Referring to FIG. 2 in combination with FIG. 1, while the system 100 may utilize tool guides of various shapes, sizes, and functionalities, the depicted tool guide 140 has a tool support 141 having an aperture 142 (see FIG. 2) for retaining, guiding, positioning, supporting, and/or locating at least one tool or guide such as the scalpel guide 160. Advantageously, the tool support 141 may be configured to guide, position, support, or locate a series of tools used in a surgical procedure, such as spinal surgery, with respect to a surgical site ST. The robotic arm 104 may be configured to help a user (e.g., a surgeon) guide, position, support, or locate the tools and/or guides along at least one planned trajectory 199 using the tool guide 140. Exemplary tools include, but are not limited to, a dilator having a dilator tip (e.g., sharp or blunt), a probe, a cutting instrument, a tap, a screw, etc. The cutting instrument may be, for example, a drill, saw blade, burr, reamer, mill, scalpel blade, or any other implement that could cut bone or other tissue and is appropriate for use in a particular surgical procedure. The tools may be secured in the tool support 141 using a locking mechanism (not shown). The locking mechanism may be a slider locking mechanism or other feature, for instance.

In some embodiments, the tool guide 140 includes the tool support 141 having the aperture 142 extending from a first face 144 of the tool support 141 to a second face 146 of the tool support 141 and has a longitudinal axis 148 that extends through a center of the aperture 142. In some embodiments, the tool support 141 can be a tube.

As described herein, some embodiments include the controller 106 that can control operation of the robotic arm 104. The controller 106 may be configured to execute the application 111 to control the robotic arm 104. In some embodiments, the application 111, in response to execution by the processor 108, can utilize trajectories (such as, tip and tail coordinates) that can be planned and/or configured remotely or locally before and/or during a surgical procedure. A trajectory that has been planned before or during the surgical procedure may be referred to herein as a “planned trajectory” such as the planned trajectory 199. In an additional or alternative aspect, in response to execution by the processor 108, the application 111 may be configured to implement one or more of the methods described herein in the controller 106 to cause movement of the robotic arm 104 according to one or more trajectories. It should be noted that for a spine surgery there are multiple planned trajectories. It would be common to have six trajectories (three pairs of two trajectories, i.e., one pair of trajectories for each vertebral body involved in the surgery). In some embodiments, four trajectories may be used for fusing two vertebral bodies together. Each planned trajectory would be identified in the application 111 and may be planned to be executed in a certain order. For instance, in an exemplary surgical procedure for fusing first and second vertebral bodies together, four planned trajectories would be used and may be identified as a first planned trajectory, a second planned trajectory, a third planned trajectory, and a fourth planned trajectory. A user, such as a surgeon, may plan to work on one side of the patient first. For example, the first planned trajectory would be directed to a first side of the first vertebral body and the second planned trajectory would be directed to a first side of the second vertebral body. The surgeon may then plan to move to the other side of the patient and the third planned trajectory would be directed to a second side of the first vertebral body and the fourth planned trajectory would be directed to a second side of the second vertebral body. It should be noted, however, that the user may plan the surgical procedure in any order and the application 111 may be programmed to cause movement of the robotic arm 104 between the planned trajectories in the planned order.

The scalpel guide 160 may be provide with a guide slot 162 sized and shaped to receive a scalpel (not shown). The scalpel guide 160 may have a body portion 164 sized and shaped to be received by the aperture 142 of the tool support 141 and secured within the tool support 141. A shoulder 166 of the scalpel guide 160 extends outwardly from the body portion 164 and may be provided to contact the first face 144 of the tool support 141 when the scalpel guide 160 is secured in the tool support 141.

The guide slot 162 is defined by a first side 166 of the scalpel guide 160, a second side 168 of the scalpel guide 160 spaced apart from the first side 166, a third side 170 of the scalpel guide 160, and a fourth side 172 of the scalpel guide 160 spaced apart from the third side 168. The guide slot 162 extends through a central region within the scalpel guide 160 and is arranged in the scalpel guide 160 such that a center of the guide slot 162 aligns with the longitudinal axis 148 of the tool guide 140 when the scalpel guide 160 is secured in the tool support 141 of the tool guide 140, the center of the guide slot 162 being equidistant between outer boundaries of the first side 166 and the second side 168 and equidistant between the third side 170 and the fourth side 172.

The non-contact marking device 171 may be provided with a body 173, a shoulder 174 extending outwardly from the body, a grip 176 adjacent to the shoulder 174 such that the shoulder 174 is positioned between the body 173 and the grip 176, a marking device 178, and a button 180.

The body 173 of the non-contact marking device 171 may be sized and shaped to be inserted into the guide slot 162 of the scalpel guide 160. In the illustrated embodiment, the body 173 is provided with a bottom face 181, a thickness T that extends from a first face 182 to a second face 184, and a width W that extends from a first side 186 to a second side 188. The thickness T and width W substantially match a thickness and width of a body of a scalpel (not shown) designed to be used with the scalpel guide 160.

The shoulder 174 may be configured to contact the scalpel guide 160 to limit a depth to which the body 173 of the non-contact marking device 171 may be inserted into the guide slot 162 of the scalpel guide 160. The body 173 may have a length corresponding to a length of the scalpel guide 160 such that when the body 173 is fully inserted into the guide slot 162, the marking device 171 is positioned adjacent to a lower end of the scalpel guide 160.

The grip 176 may be provided to facilitate a user in inserting and/or removing the non-contact marking device 171 from the scalpel guide 160. The grip 176 may be textured to assist the user in gripping and/or manipulating the non-contact marking device 171.

The button 180 may be attached to, placed in, or otherwise formed in the grip 176. The button 180 may be configured to turn the marking device 178 on and/or off by, for instance, pressing the button 180. The button 180 may be any type of switch known in the art. By way of illustration and not limitation, the button 180 may be a pushbutton, a selector switch, a proximity switch, or a pressure switch, for instance.

The non-contact marking device 171 may further be provided with other electronic mechanisms such as a power source, e.g., battery and wiring that are not shown that may electrically connect the button 180 and the marking device 178. Such electronic mechanisms are known in the art and, in the interest of brevity, will not be described further herein. The electronic mechanisms may be disposed in the body 173 of the non-contact marking device 171 using means known in the art.

The marking device 178 may be any type of device known in the art capable of projecting, emitting, and/or transmitting a focused beam of light in a wavelength visible to a human. For instance, the marking device 178 may be a laser. The marking device 178 may be disposed in a center of the bottom face 181 of the body 173 such that when the non-contact marking device 171 is inserted in the guide slot 162 of the scalpel guide 160 which is inserted in the aperture 142 of tool guide 140, the marking device 178 is coaxially aligned with the longitudinal axis 148 of the tool guide 140. The marking device 178, when turned on or switched to an on state, projects, emits, and/or transmits the focused beam of light that may be viewed on an opaque surface such as human skin as a marker.

In the illustrated embodiment, the body 173 forms a generally rectangular prism shape extending from the bottom face 181 to the shoulder 174 with the first face 182 parallel to and separated a first predetermined distance from the second face 184, the first predetermined distance being the thickness T, and the first side 186 parallel to and separated a second predetermined distance from the second side 188, the second predetermined distance being the width W. However, it should be noted that the non-contact marking device 170 may be adapted to work with any contact-type marking device designed for use with a surgical robot. In such embodiments, the body 173 of the non-contact marking device 171 may be adapted to match a shape and/or design of a guide for the contact-type marking device.

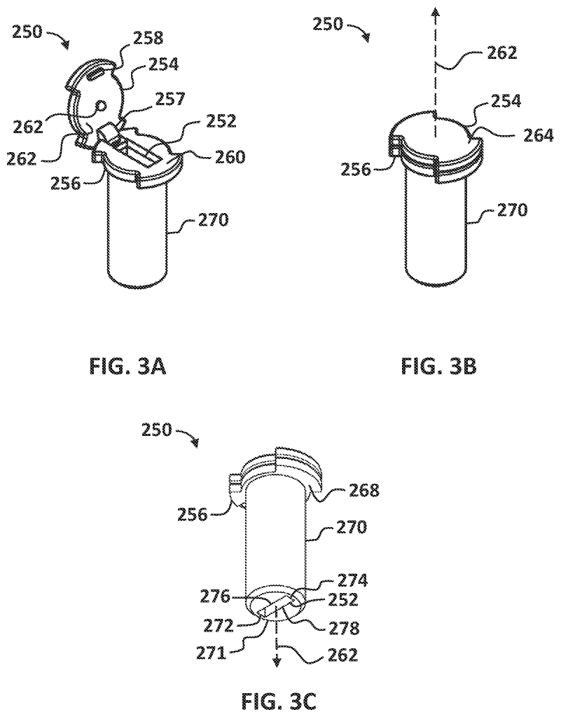

Referring now to FIGS. 3A, 3B, and 3C, shown therein is a non-contact marking device 250 comprising a scalpel guide 252, a top 254, a shoulder extending outwardly from the scalpel guide 252, a hinge 257, a button 258, and a marking device 262. The non-contact marking device 250 may be provided with the scalpel guide 252 configured to receive a body of a scalpel (not shown) when the top 254 of the non-contact marking device 250 is in an open position as shown in FIG. 3A. The top 254 may be hingedly connected to the shoulder portion 256 by the hinge 257 or other suitable mechanism that allows the top 254 to be moved between the open position and a closed position (shown in FIG. 3B). When in the closed position, the button 258 may be depressed or otherwise engaged by contacting a top face 260 of the shoulder portion 256. When the button 258 is engaged, the marking device 262 may be turned on and project or emit a light in a wavelength visible to a human. For instance, the marking device 262 may be a laser.

The top 254 may be provided with a lower surface 262 and an upper surface 264 spaced a predetermined distance apart.

When the top 254 is in the closed position, the marking device 262 is positioned in line with a central axis 264 of the non-contact marking device 250. When the non-contact marking device 250 is inserted into the aperture 142 of the tool support 141, for instance, and the top 254 is in the closed position, the central axis 264 is coaxially aligned with the longitudinal axis 148 of the tool support 141 which will align the marking device 262 with a planned trajectory for a surgery.

The scalpel guide 252 of the non-contact marking device 250 may also have a body 270 that extends from a bottom 271 to a lower face 268 of the shoulder 256. The body 270 is generally cylindrically shaped in the illustrated embodiment and sized and shaped to be received and secured within the aperture 142 of the tool support 141, for instance. However, it should be noted that the non-contact marking device 250 may be adapted to work with any contact-type marking device designed for use with a surgical robot. In such embodiments, the body 270 of the non-contact marking device 250 may be adapted to match a shape and/or design of a tool guide for the contact-type marking device.

The shoulder 256 of the non-contact marking device 250 is provided with the lower face 268 which may contact the first face 144 of the tool support 141 when the non-contact marking device 250 is positioned in the aperture 142 of the tool support 141 and act as a stop limiting a distance the non-contact marking device 250 may be inserted into the aperture 142 of the tool support 141.

The guide slot 252 extends through the body 270 and the shoulder 256 and is defined by a first side 272, a second side 274 spaced apart from the first side 272, a third side 276, and a fourth side 278 spaced apart from the third side 276. The guide slot 252 is arranged in the non-contact marking device 250 such that a center of the guide slot 252 aligns with the longitudinal axis 148 of the tool support 141 when the non-contact marking device 250 is secured in the tool support 141, the center of the guide slot 252 being equidistant between the first side 272 and the second side 274 and equidistant between the third side 276 and the fourth side 278.

The button 258 may be attached to, placed in, or otherwise formed in the bottom surface 262 of the top 254. The button 258 may be configured to turn the marking device 262 on and/or off by, for instance, pressing the button 258. The button 258 may be any type of switch known in the art. By way of illustration and not limitation, the button 258 may be a momentary switch, pushbutton switch, a selector switch, a proximity switch, or a pressure switch, for instance. When the top 254 is in the closed position, the button 258 may contact the top face 260 of the shoulder 256 which engages the button 258 causing the button 258 to turn the marking device 262 on. When the top 254 is in the open position, the button 258 is not engaged causing the button 258 to turn the marking device 262 off.

The non-contact marking device 250 may further be provided with other electronic mechanisms such as a power source, e.g., battery and wiring that are not shown that may electrically connect the button 258 and the marking device 262. Such electronic mechanisms are known in the art and, in the interest of brevity, will not be described further herein. The electronic mechanisms may be disposed in the top 254 of the non-contact marking device 250 using means known in the art.

The marking device 262 may be any type of device known in the art capable of projecting, emitting, and/or transmitting a focused beam of light in a wavelength visible to a human. For instance, the marking device 262 may be a laser. The marking device 262 may be disposed in a center of the top 254 of the non-contact marking device 250 such that when the non-contact marking device 250 is inserted in the aperture 142 of tool support 141, for instance, and the top 254 is in the closed position, the marking device 262 is coaxially aligned with the longitudinal axis 148 of the tool support 141. The marking device 262, when turned on or switched to an on state, projects, emits, and/or transmits the focused beam of light that may be viewed on an opaque surface such as human skin as a marker.

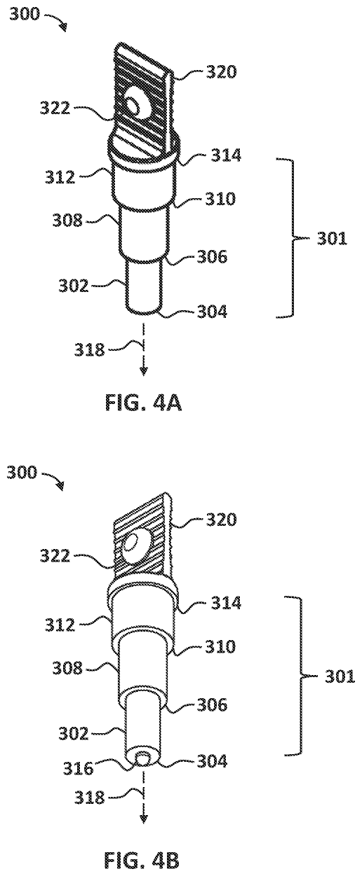

Referring now to FIGS. 4A and 4B, shown therein are perspective views of a non-contact marking device 300 having a marking device integrated into a body sized to be received in the aperture 142 of the tool support 141. In some embodiments, the non-contact marking device 300 is configured to be used with different sizes of apertures 142 within tool supports 141. In these embodiments, the non-contact marking device 300 may generally be described as a stepped device with a body 300 having a first body portion 302 having a first diameter that extends from a bottom face 304 to a first shoulder 306, a second body portion 308 having a second diameter larger than the first diameter extending from the first shoulder 306 to a second shoulder 310, and a third body portion 312 having a third diameter larger than the second diameter extending from the second shoulder 310 to a third shoulder 314. A marking device 316 may be disposed in the bottom face 304 aligned with a central axis 318 of the non-contact marking device 300.

The first diameter, second diameter, and third diameter may be sized to fit different diameter tool supports 141. For instance, the first diameter may be 10 mm, the second diameter may be 14 mm, and third diameter may be 16 mm. It should be noted that these measurements are provided for illustration purposes only and the non-contact marking device 300 may be provided with first diameter, second diameter, and third diameters, etc. that are sized and shaped to fit a particular size of tool support 141. Further, the first body portion 302, second body portion 304, and third body portion 306 are shown as generally cylindrically shaped for illustration purposes only and it should be appreciated that the first body portion 302, second body portion 304, and third body portion 306 may be provided having any shape configured to be inserted into the aperture 142 of the tool support 141.

The non-contact marking device 300 may further be provided with a grip 320 and a button 322 which may be disposed in the grip 320. The grip 320 may be provided to facilitate a user in inserting and/or removing the non-contact marking device 300 from a tool guide such as tool guide 140. The grip 320 may be provided having texture to assist the user in gripping and/or manipulating the non-contact marking device 300.

The button 322 may be attached to, placed in, or otherwise formed in the grip 320. The button 322 may be configured to turn the marking device 316 on and/or off by, for instance, a user pressing the button 322. The button 322 may be any type of button or switch known in the art. By way of illustration and not limitation, the button 322 may be a pushbutton, a selector switch, a proximity switch, or a pressure switch, for instance.

The non-contact marking device 300 may further be provided with other electronic mechanisms such as a power source, e.g., battery and wiring that are not shown that may electrically connect the button 322 and the marking device 316. Such electronic mechanisms are known in the art and, in the interest of brevity, will not be described further herein. The electronic mechanisms may be disposed in the first body portion 302, second body portion 304, third body portion 306, and/or grip 320 of the non-contact marking device 300 using means known in the art.

The marking device 316 may be any type of device known in the art capable of projecting, emitting, and/or transmitting a focused beam of light in a wavelength visible to a human. For instance, the marking device 316 may be a laser. The marking device 316 may be disposed in a center of the bottom face 304 of the non-contact marking device 300 such that when the non-contact marking device 300 is inserted in the aperture 142 of tool guide 140, for instance, the marking device 316 is coaxially aligned with the longitudinal axis 148 of the tool guide 140. The marking device 316, when turned on or switched to an on state, projects, emits, and/or transmits the focused beam of light that may be viewed on an opaque surface such as human skin as a marker.

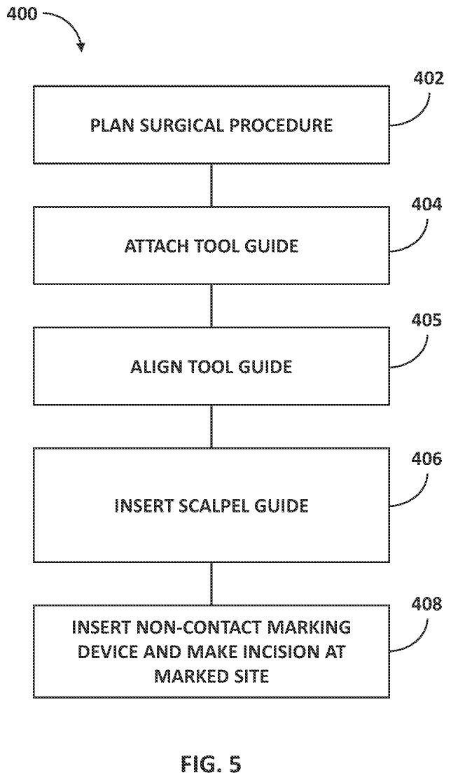

Referring now to FIG. 5, shown therein is an illustrative process 400 for using a non-contact marking device in a surgical procedure (e.g., pursuant to a treatment plan) which may be employed with a computer-assisted surgical system (e.g., such as the computer-assisted surgical system 100 of FIG. 1 having the robotic arm 104, the controller 106, and the navigational system 120). For example, the surgical procedure may involve a patient's spine, such as placement of screws in one or more pedicles of a patient's vertebrae. By way of a non-limiting example, the surgical procedure may employ a drill, tap, and screw technique, such as may be required as part of a transforaminal lumbar interbody fusion (TLIF) procedure. A series of tools may be required by the surgical procedure. One example of a procedure is a posterior pedicle screw placement for posterior stabilization which is often performed together with an interbody procedure (e.g., placement of a cage).

In a first step 402, the processor 108 of the controller 106 may be programmed to enable a user (e.g., a surgeon) to locate an intended position of a surgical implant or tool. For instance, at least one trajectory to access anatomical structures of a patient. For instance, each trajectory may be planned using imaging of the patient anatomy (e.g., magnetic resonance imaging scans of the lower lumbar region of the spine) that have been used to create anatomical computer models, data from previous surgical procedures and/or previously-performed surgical techniques (e.g., data recorded by the system 100 while forming pilot holes that are subsequently used to facilitate installation of an anchor), and the like. In some embodiments, the user may program a desired point of insertion and trajectory for a surgical instrument to reach a desired anatomical target within or upon the body B of the patient. In some embodiments, the desired point of insertion and trajectory can be planned on the anatomical computer model, which in some embodiments, can be displayed on the external device 114. In some embodiments, the user can plan the trajectory and desired insertion point (if any) on a computed tomography scan (hereinafter referred to as “CT scan”) of the patient. In some embodiments, the CT scan can be an isocentric C-arm type scan, an O-arm type scan, or intraoperative CT scan as is known in the art. However, in some embodiments, any known 3D image scan can be used in accordance with the embodiments of the invention described herein. The at least one trajectory planned as described in step 402 may be referred to throughout as a “planned trajectory” such as the planned trajectory 199.

At step 404, the tool guide 140, e.g., having the tool support 141, e.g., a connector or coupler, that is adapted to receive a plurality of tools (e.g., different tools sequentially) is supported (e.g., attached or mounted) to the distal end 107b or other location on the robotic arm 104 of the computer-assisted surgical system 100. The tool guide 140 may be coupled to the robotic arm 104, for example, via an end plate locked by a lever or other coupling means known in the art such as screws, bolts, threaded connection, and the like.

In step 405, alignment of the tool guide to the planned trajectory 199 is performed. In one example, after the tool guide 140 is connected to an active robotic arm such as robotic arm 104, navigational assessments using the controller 106 and associated navigational system 120 may be performed to ensure alignment of the tool guide 140. Alignment may be performed with no tool 150 in the tool guide 140, with the tool 150 being a referencing tool in the tool guide 140, or with an initial tool 150 of the surgical procedure in the tool guide 140. In some embodiments, the robotic arm 104 is only aligned once to a planned trajectory (for example, per pedicle screw insertion procedure). With respect to the tool guide 140, mounting may only need to be performed once, but with respect to the tool 150 in the surgical procedure (e.g., each tool 150 in the surgical procedure), a mounting and alignment step may be repeated at each tool change and/or distal tip change. A common reason for realignment is a detected deviation from the planned trajectory 199 due to applied forces on the surgical system 100.

At step 406, the scalpel guide 160 is placed in the tool support 141 of the tool guide 140 and secured in place. Optionally, step 406 may be performed with respect to alignment of the tool guide 140 (e.g., at a desired trajectory, position, and/or orientation). The robotic arm 104 may navigate to a starting position (a system with an active robot arm) or may be guided to the starting position (a system with a passive arm with an active tool guide) by the user (e.g., a surgeon). The navigational system 120 and/or the controller 106 may store the position (e.g., a three-dimensional position). A cutting trajectory may be displayed on the external device 114, along with imaging of the patient anatomy, etc.

At step 408, the non-contact marking device 170 may be inserted in the guide slot 162 of the scalpel guide 160 and turned on causing the marking device 178 to project, emit, and/or transmit a visible light. When the tool guide 140 is aligned with the planned trajectory 199, the marking device 178 projects the visible light onto the body B of the patient marking a point at an intersection of the planned trajectory 199 and the body B of the patient which may be referred to as an entry point. This allows the user to open an incision along the planned trajectory 199 to access the surgical site ST. Once the incision at the surgical site ST is complete, patient anatomical structures such as a bone surface, are accessible. The navigational system 120 and/or the controller 106 may store a position (e.g., a three-dimensional position) of the robotic arm 104 and/or the tool guide 140. For example, this position may include an incision boundary, or an incision depth determination. The scalpel guide 160 and the non-contact marking device 170 may be removed from the tool guide 140.

Optionally, steps 406 and 408 may be combined by, for instance, inserting the non-contact marking device 170 into the guide slot 162 of the scalpel guide 160 before placing the scalpel guide 160 into the tool support 141.

When the surgical procedure includes multiple planned trajectories, such as a surgical procedure requiring placement of multiple pedicle screws, a first pedicle screw may be placed along a first planned trajectory. After the first pedicle screw is placed, the user may move the robotic arm 104 from a current position, which may be on the first planned trajectory, to a second planned trajectory for placing a second pedicle screw. The workflow 400 may be repeated for opening an incision site for the second pedicle screw beginning at step 405, for instance.

As can be appreciated, the presently described embodiments may also be applicable to other surgical procedures such as cervical procedures, etc.

From the above description, it is clear that the inventive concept(s) disclosed herein are well adapted to carry out the objects and to attain the advantages mentioned herein, as well as those inherent in the inventive concept(s) disclosed herein. While the embodiments of the inventive concept(s) disclosed herein have been described for purposes of this disclosure, it will be understood that numerous changes may be made and readily suggested to those skilled in the art which are accomplished within the scope and spirit of the inventive concept(s) disclosed herein.

What is claimed is:

1. A non-contact marking device, comprising:
a body having a bottom face, a first face, a second face separated a first predetermined distance from the first face, a first side, and a second side separated a second predetermined distance from the first side, the body sized and shaped to be at least partially inserted into a guide slot of a scalpel guide;
a shoulder extending outwardly from the body and formed a predetermined distance from the bottom face of the body and configured to limit a distance the body may be at least partially inserted into the guide slot of the scalpel guide; and
a marking device configured to emit a light, the marking device configured to project light from the bottom face of the body such that the light projected by the marking device is coaxially aligned with a longitudinal axis of a tool guide of a surgical system when the non-contact marking device is at least partially inserted in the guide slot of the scalpel guide while the scalpel guide is inserted in the tool guide of the surgical system.

2. The non-contact making device of claim 1, further comprising a button electrically connected to the marking device and configured to switch the marking device from an off state wherein the marking device does not emit the light to an on state wherein the marking device emits the light.

3. The non-contact marking device of claim 1, wherein the marking device comprises a laser.

4. The non-contact marking device of claim 1, wherein the body has a generally rectangular prism shape.

5. The non-contact marking device of claim 1, further comprising a power source supplying power to the marking device.

6. The non-contact marking device of claim 1, wherein the light includes a wavelength or range of wavelengths visible to a human eye.

7. A non-contact marking device, comprising:
a body having a lower face and an upper face, the body sized and shaped to be at least partially inserted into an aperture of a tool guide of a surgical system, the body having a guide slot formed in and extending through the body from the upper face to the lower face, the guide slot sized and shaped to receive a body of a scalpel, the body of the non-contact marking device having;
a shoulder extending outwardly from the body;
a top having an upper surface and a lower surface spaced a predetermined distance apart and connected to at least one of the body and the shoulder;
a marking device supported by the top configured to emit a light through the guide slot, the marking device disposed in the lower surface of the top; and
a button electrically connected to the marking device and configured to switch the marking device from an off state wherein the marking device does not emit light to an on state wherein the marking device emits the light.

8. The non-contact marking device of claim 7, wherein the top is hingedly connected to the shoulder such that the top may be moved between an open position and a closed position.

9. The non-contact marking device of claim 8, wherein the button is supported by the lower surface of the top and when the top is in the closed position the lower surface of the top and the button are in contact with a top face of the shoulder and the marking device is in the on state.

10. The non-contact marking device of claim 7, wherein the light is in a wavelength or range of wavelengths visible to a human eye.

11. A robotic surgical system comprising:
a robotic arm;
a tool guide supported by the robotic arm, the tool guide comprising a tool support having a first end, a second end, an aperture extending through the tool support from the first end to the second end, and a longitudinal axis extending through a center of the aperture from the first end to the second end; and
a controller in communication with the robotic arm, the controller having a non-transitory computer readable memory and a processor, the non-transitory computer readable memory storing at least one planned trajectory associated with a surgical procedure and processor executable instructions that, when executed, cause the processor to pass a first signal to the robotic arm causing the robotic arm to position the tool support a distance from a patient with the longitudinal axis of the tool support substantially coaxially aligned with the at least one planned trajectory; and
a non-contact marking device comprising:
a body having a first body portion having a first diameter extending from a bottom face to a first shoulder and a second body portion having a second diameter that is larger than the first diameter extending from the first shoulder to a second shoulder, the body positioned within the aperture of the tool support, the body having a central axis; and
a marking device configured to emit a light in a wavelength visible to a human eye, the marking device configured to emit light from the bottom face of the body such that the marking device is coaxially aligned with the central axis extending through a center of the body.

12. The robotic surgical system of claim 11, further comprising a button electrically connected to the marking device and configured to switch the marking device from an off state wherein the marking device does not emit the light to an on state wherein the marking device emits the light.

13. The robotic surgical system of claim 11, wherein the body of the non-contact marking device further comprises a third body portion having a third diameter that is larger than the second diameter, the third body portion extending from the second shoulder to a third shoulder.
